(19) United States
(12) Patent Application Publication
(10) Pub. No.: US 2026/0041345 A1
(43) Pub. Date: Feb. 12, 2026

(54) SYSTEMS AND METHODS FOR DIAGNOSIS AND TREATMENT OF MENTAL ILLNESSES IN VIRTUAL REALITY ENVIRONMENTS

(71) Applicant: Nitya Jani, Frederick, MD (US)

(72) Inventor: Nitya Jani, Frederick, MD (US)

(21) Appl. No.: 19/290,949

(22) Filed: Aug. 5, 2025

Related U.S. Application Data
(60) Provisional application No. 63/680,463, filed on Aug. 7, 2024.

(51) Int. Cl. A61B 5/16; A61B 5/00; G16H 40/67; G16H 50/20

(52) U.S. Cl. CPC A61B 5/163; A61B 5/4842; A61B 5/742; A61B 5/7475; G16H 40/67; G16H 50/20

(57) ABSTRACT

A device for diagnosing one or more mental illnesses using a virtual reality (VR) environment in which a first user and a second user are present, may receive, from a head worn device (HWD) of the first user, first data representing a first motion of the first user in the VR environment. The device may determine, based on the first data, that the first motion of the first user relates to a first symptom of the one or more mental illnesses, receive, from the HWD of the first user, second data representing at least one of an intensity, a boundary, or a frequency of the first motion of the first user, determine, based on the second data, a severity level of the first symptom, and control a wearable device of the second user to generate a first haptic output corresponding to the severity level of the first symptom.

### CROSS-REFERENCE TO RELATED APPLICATIONS

This application claims the benefit of and priority to U.S. Provisional Application No. 63/680,463, filed on Aug. 7, 2024, which is incorporated herein by reference in its entirety for all purposes.

### TECHNICAL FIELD

The present disclosure relates to systems and methods for diagnosis and/or treatment of one or more mental illnesses and more particularly to systems and methods for diagnosing and/or treating one or more mental illnesses using a virtual reality (VR) environment.

### BACKGROUND

Play therapy is a certified form of psychotherapy primarily utilized with children. It involves the use of toys, figurines, art supplies, musical instruments, and similar materials as mediums for expression. Children, like adults, experience hardship and trauma; however, they often lack the linguistic capacity to comprehend and articulate these experiences verbally. Play therapy addresses this limitation by providing an alternative mode of communication that bridges the expressive gap commonly encountered in children.

Within the context of play therapy, various thematic approaches are utilized. Among these, three thematic approaches may be employed: modeling, transference, and social skill development. Modeling entails the use of toys to simulate real-life scenarios, enabling the child to externalize and represent complex situations through symbolic play. Transference allows the child to project internal emotional states onto representative avatars—such as dolls or animals—thereby facilitating emotional distancing and enabling the child to engage with their struggles from a safer psychological vantage point. Social skill development involves the structured use of play to instill essential interpersonal behaviors, including turn-taking, etiquette, and other forms of behavioral reinforcement.

Play therapy sessions are conducted between a trained play therapist and one or more clients, typically children. Due to the resource-intensive and interactive nature of these sessions, remote or online implementation presents significant challenges, including limited access to necessary materials and reduced therapeutic efficacy.

### SUMMARY

Implementations of the present disclosure relate to a system and a method for diagnosis and/or treatment of one or more mental illnesses and more particularly to systems and methods for diagnosing and/or treating one or more mental illnesses using a virtual reality (VR) environment.

In some implementations of the present disclosure, a device for diagnosing one or more mental illnesses using a virtual reality (VR) environment in which a first user and a second user are present, may include one or more processors. The one or more processors may be configured to receive, from a head worn device (HWD) of the first user, first data representing a first motion of the first user in the VR environment. The one or more processors may be configured to determine, based on the first data, that the first motion of the first user relates to a first symptom of the one or more mental illnesses. The one or more processors may be configured to receive, from the HWD of the first user, second data representing at least one of an intensity, a boundary, or a frequency of the first motion of the first user. The one or more processors may be configured to determine, based on the second data, a severity level of the first symptom. The one or more processors may be configured to control a wearable device of the second user to generate a first haptic output corresponding to the severity level of the first symptom.

In some implementations of the present disclosure, a method for diagnosing one or more mental illnesses using a virtual reality (VR) environment in which a first user and a second user are present, may include receiving, by one or more processors, from a head worn device (HWD) of the first user, first data representing a first motion of the first user in the VR environment. The method may include determining, by the one or more processors based on the first data, that the first motion of the first user relates to a first symptom of the one or more mental illnesses. The method may include receiving, by the one or more processors, from the HWD of the first user, second data representing at least one of an intensity, a boundary, or a frequency of the first motion of the first user. The method may include determining, by the one or more processors based on the second data, a severity level of the first symptom. The method may include controlling, by the one or more processors, a wearable device of the second user to generate a first haptic output corresponding to the severity level of the first symptom.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1 is a block diagram illustrating an example of a system environment for a virtual reality (VR) mental illness diagnosis system, according to some implementations.

FIG. 2 is a block diagram illustrating an example of a computing system according to some implementations.

FIG. 3 is an example virtual playroom (e.g., a virtual stuffed animal tent) in a virtual play therapy platform, according to some implementations.

FIG. 4 is an example virtual playroom (e.g., a virtual dollhouse section) in a virtual play therapy platform, according to some implementations.

FIG. 5 is an example virtual playroom (e.g., a virtual nurturing section) in a virtual play therapy platform, according to some implementations.

FIG. 6 is an example virtual playroom (e.g., a virtual sports pen) in a virtual play therapy platform, according to some implementations.

FIG. 7 is an example virtual playroom (e.g., a virtual cityscape) in a virtual play therapy platform, according to some implementations.

FIG. 8 is an example virtual playroom (e.g., a virtual kitchen) in a virtual play therapy platform, according to some implementations.

FIG. 9 is an example virtual playroom (e.g., a virtual game room) in a virtual play therapy platform, according to some implementations.

FIG. 10 is an example virtual playroom (e.g., a virtual music section) in a virtual play therapy platform, according to some implementations.

FIG. 11 is an example virtual playroom (e.g., a virtual general toys section) in a virtual play therapy platform, according to some implementations.

FIG. 12 is an example virtual playroom (e.g., a virtual sand tray section) in a virtual play therapy platform, according to some implementations.

FIG. 13 is an example virtual playroom (e.g., a virtual art table section) in a virtual play therapy platform, according to some implementations.

FIG. 14 is an example virtual playroom (e.g., a virtual shopping section) in a virtual play therapy platform, according to some implementations.

FIG. 15 is a flowchart illustrating an example methodology for diagnosing one or more mental illnesses using a VR environment, according to some implementations.

### DETAILED DESCRIPTION

According to certain aspects, implementations in the present disclosure relate to a system and a method for diagnosis and/or treatment of one or more mental illnesses and more particularly to systems and methods for diagnosing and/or treating one or more mental illnesses using a virtual reality (VR) environment.

Several open-source platforms currently offer virtual playrooms that operate within a two-dimensional interface. These environments typically feature movable two-dimensional representations of toys or stickers. However, they do not support manipulation of objects within a three-dimensional spatial framework, nor do they permit user navigation through the virtual space in a manner that replicates real-world movement. While VR environments are available, they are not specifically designed to support the therapeutic objectives of play therapy.

Digital communication technologies are in use by therapists and mental health professionals for the diagnosis and treatment of mental illness. These include video conferencing applications such as Zoom and dedicated therapy platforms like Grow Therapy. Such tools facilitate verbal interaction between clients and therapists, enabling remote delivery of talk-based therapeutic services.

Technological applications have been integrated into various therapeutic approaches. As noted by Daniel and Panchanathan (2019), these include implementations in gaming and play, emotional regulation, stimulation therapy, and distributed touch therapy. Within the domain of games and play, robotic systems have been developed to provide companionship and to enable physiological sensing for control within virtual environments. These systems exhibit enhanced expressive capabilities, particularly beneficial for users with limited verbal communication. The adoption of general teletherapy has increased significantly following the COVID-19 pandemic. However, the virtual delivery of play therapy remains challenging due to its reliance on physical tools and toys. A fundamental therapeutic component of play therapy lies in the child's ability to physically navigate a playroom, select objects, and engage with them through manipulation. This experiential aspect is not easily replicated through telehealth platforms or for children who are confined to a bed.

To solve the above-noted problems, according to certain aspects, a system for diagnosing and/or treating one or more mental illnesses using a VR environment (e.g., VR mental illness diagnosis/treatment system) can provide a digital method for diagnosis and treatment of mental illness or general emotional struggles. In some implementations, the system can provide a means of conducting virtual play therapy through software that simulates the therapeutic experience of a child navigating a playroom, selecting toys, and engaging with them. In some implementations, a method for diagnosing and/or treating one or more mental illnesses using a VR environment (e.g., VR mental illness diagnosis/treatment method) can enable the facilitation of play therapy sessions via an online platform that features a virtual environment populated with toys, tools, and other expressive elements (e.g., video/audio chat between a client and a therapist), serving as the medium for therapeutic interaction.

In some implementations, the system/method can provide a virtual environment or virtual platform for play therapy using the technologies of virtual reality and/or teletherapy. The system can provide a capability to conduct highly realistic play therapy sessions in a digital format (e.g., virtual environment). Within this VR environment, clients (e.g., children) can have full control over their movements, choices of objects, and modes of play. The three-dimensional (3D) structure of the platform can allow therapists to implement research-based practices in play therapy and general child therapy. In contrast, two-dimensional (2D) environments lack the immersive quality necessary to create a sense of spatial presence and do not permit the same level of object manipulation afforded by 3D representations.

In some implementations, the system/method can provide a digital (e.g., virtual) therapeutic space that transcends conventional verbal interaction. The system/method can replicate a physical playroom, allowing clients to engage with a virtual play in a manner analogous to real-world play. The use of virtual play spaces (e.g., virtual playrooms) can distinguish the system/method from standard digital tools commonly used in therapy, such as videoconferencing platforms. The system/method can be used by a trained mental health professional and one or more clients, with both parties having the option to interact with the virtual environment. Using the system/method, verbal communication may occur concurrently with engagement in the virtual space, and the therapist can utilize the virtual environment as a medium through which general therapeutic techniques and methodologies are applied.

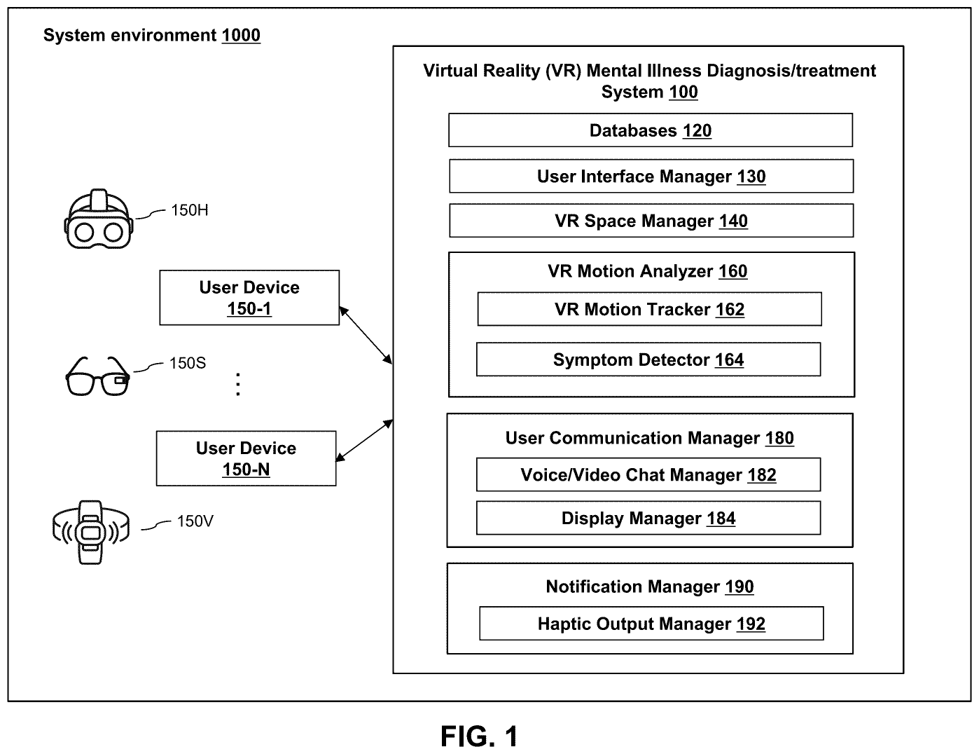

FIG. 1 is a block diagram illustrating an example of a system environment 1000 for a system for diagnosing and/or treating one or more mental illnesses using a VR environment (e.g., a VR mental illness diagnosis/treatment system 100) according to some implementations. The system 100 may be coupled or paired with a plurality of user devices 120-1 to 120-N.

In some implementations, the plurality of user devices may include VR user devices such as head-worn devices (HWDs), motion sensors, wearable devices, VR controllers. The HWDs may include head-mounted display (HMD) 150H, or HMD integrated into a helmet or glasses frames (e.g., smart glasses 150S). The motion sensors may include sensors that can track eyes, head, hand, or body movements to adjust the virtual view in real time. The wearable devises (or wearables) can provide tactile feedback (haptics) so users can feel virtual objects. The wearable devices can generate mechanical stimuli (vibrations, forces, or motions) that interact with the user's skin or muscles, using actuators (e.g., vibration motors 150V, piezoelectric elements, or air bladders) to create these sensations. The wearable devices can vary intensity, frequency and/or duration to simulate different textures, impacts, and/or resistance. Examples of the wearable devices may include smartwatches, haptic suits, haptic gloves, etc. The VR controllers can enable interaction with virtual elements through buttons, triggers, or gestures.

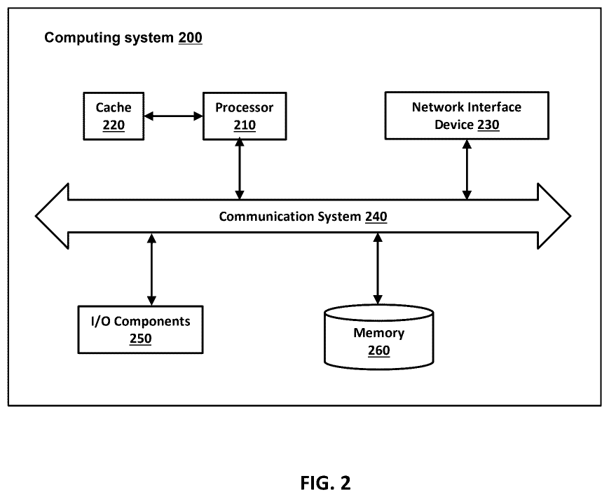

In some implementations, the system 100 may be connected to the plurality of user devices 120-1 to 120-N via a network. Here, the network may be a Local Area Network (“LAN”), a wide area network (“WAN”), a wireless network, and/or the Internet, among others. The wireless network may be the IEEE 802.11 protocols, near field communication (NFC), Bluetooth, ANT, or any other wireless protocol, among others. Each of the system 100 and the plurality of user devices 150-1 to 150-N may have configurations similar to those of computing system 200 in FIG. 2.

In some implementations, the system 100 may include one or more databases 120, a user interface manager 130, a VR space manager 140, a VR motion analyzer 160, a user communication manager 180 and/or a notification manager 190, which will be described in more details in the following sections. In some embodiments, at least one or more of the user interface manager 130, the VR space manager 140, the VR motion analyzer 160, the user communication manager 180 and/or the notification manager 190 may be implemented with a circuit (e.g., circuitry of a FPGA, CPU, GPU or other processing circuits implemented using electronic circuits), a subroutine in a program stored in memory (e.g., EPROM, EEPROM, SDRAM, and flash memory devices, CD ROM, DVD-ROM, or Blu-Ray® discs and the like) and executable by a processor (e.g., CPU, GPU and the like), or the like. The VR motion analyzer 160 may include a motion tracker 162, a symptom detector 164, and/or symptom level detector 166. The user communication manager 180 may include a voice/video chat manager 182 and/or a display manager 184. The notification manager 190 may include a haptic output manager 192.

In some implementations, the system 100 may include one or more databases 120 to store data managed or used by one or more of the user interface manager 130, the VR space manager 140, the VR motion analyzer 160, the user communication manager 180 and/or the notification manager 190 (e.g., data relating to user interfaces, virtual spaces such as virtual playrooms, virtual objects/items, user's motions in virtual spaces, mental illnesses and symptoms, information of users, information of user devices, etc.). In some implementations, the user interface manager 130 can provide menus for accessing (e.g., logging-in), creating, adjusting, modifying, repositioning, rearranging, and/or deleting virtual spaces and virtual objects/items. The user interface manager 130 can also provide menus for saving configurations of virtual spaces/objects and reopening the configurations. In some implementations, the VR space manager 140 may create, adjust, modify, reposition, and/or delete virtual spaces and virtual objects/items, in response to users' motions or manipulations. In some implementations, the VR motion analyzer 160 may track or detect users' motions in virtual spaces (e.g., motion tracker 162), and/or analyze the motions to detect or determine symptoms of one or more mental illnesses and symptom levels thereof (e.g., symptom detector 164). In some implementations, the user communication manager 180 may provide means or tools for communicating between users using a voice chat and/or a video chat (e.g., voice/video chat manager 182) and/or sharing a display (e.g., display manager 184). In some implementations, the notification manager 190 may notify or inform users of information on the motion of a user using various methods including text, voice, video, and/or haptic feedback (e.g., haptic output manager 192). For example, the notification manager 190 may generate a report detailing the duration spent in each zone, which is then made available to the therapist. In another example, the notification manager 190 may notify a user of symptoms or symptom levels of mental illnesses and/or diagnosis results of mental illnesses by generating/outputting a haptic feedback to the user.

In some implementations, the system (e.g., VR space manager 140) can provide a digital therapeutic environment in which a mental health professional may configure the virtual space prior to client interaction. For example, the VR space manager 140 can allow a therapist or mental health professional to reposition or remove objects within the virtual environment before the client accesses the virtual environment.

In some implementations, the system (e.g., VR space manager 140) can allow users to manipulate objects within the VR space. The system can provide access to the virtual environment through either a mobile device or a web-based platform, allowing for flexible entry points. The VR space manager 140 can support simultaneous participation by both the mental health professional and the client within the same virtual space (e.g., the same playroom). This can be facilitated or implemented through a multiplayer VR framework that initiates a shared server instance, enabling both parties to enter the environment concurrently. Each participant can be represented by an avatar and equipped with a microphone instance to support real-time voice communication.

In some implementations, the system (e.g., user interface manager 130, databases 120) can allow the mental health professional to preserve specific configurations (e.g., configuration instances) of the virtual space. These instances may be stored on a server or database (e.g., databases 120) and reopened at a later time through a user interface menu. The user interface manager 130 can provide user interfaces for enabling categorization, naming, and reorganization of saved instances, thereby supporting structured therapeutic workflows.

In some implementations, the system (e.g., user interface manager 130) can allow clients (or therapists) to personalize the virtual environment. This includes modifying visual elements such as wall colors and object placements. The user interface manager 130 can provide a user interface menu (e.g., menu implemented through Unity's canvas system) that presents selectable materials that are applied to designated surfaces upon interaction. The user interface manager 130 can allow clients to add or remove objects from the environment using a similar interface, where object visibility is toggled through menu selections.

In some implementations, the system (e.g., user interface manager 130) can allow for both remote use (e.g., online) and in-person use (e.g., offline). The system can allow the therapist to project the virtual environment onto a device (e.g., the Meta Quest application), enabling co-located interaction.

In some implementations, the system (e.g., user interface manager 130, VR space manager 140) can be adaptable for use by child life specialists and other professionals working with children. For example, the system can allow the virtual environment to incorporate life-sized representations of medical equipment, such as MRI machines, to familiarize children with clinical procedures. The system can allow children to rotate and translating specific parts of these assets to replicate the movement of real-world machines, enhancing realism and educational value. In this manner, the system/method can be suitable for deployment in various child-centered environments, including hospitals and foster care centers.

In some implementations, the system (e.g., VR space manager 140) can provide interactive assets designed/configured to simulate physical transformation. For example, the VR space manager 140 can create an object (as put together) as well as pieces of the object (as broken). The VR space manager 140 can allow individual children of a prefab to transform the put-together object into the broken one on throw (by using animation), and after a predetermined seconds (e.g., 2 seconds), transform the broken object back into the put-together one (by using animation). The term “prefabs” may refer to the objects or assets that can be saved in specific ways in a real-time VR/game development engine (e.g., Unity®). For example, a main object can have parts called “children” and the children follow the parent (in the sense that if a user moves the parent, the children move too). The children can have different functionality than the parent, but when the user breaks an object (parent), the children are still part of that object and the separate pieces cannot be manipulated individually.

In some implementations, the system (e.g., VR space manager 140) can provide avatar customization through a user interface that allows selection of clothing and physical features (e.g., user interface manager 130). In some implementations, a prefab associated with a particular avatar can be attached to an HWD of a client who can make selections to dynamically modify the particular avatar.

In some implementations, the system (e.g., VR space manager 140) can provide painting/erasing features (e.g., using a line renderer) in a VR environment. The line renderer can track a movement of a controller (which is held by a user) while the trigger (e.g., part of the VR controller) is held, enabling users to draw on designated surfaces. In some implementations, the line renderer can track a movement of a controller while the trigger is not held.

In some implementations, the system (e.g., VR space manager 140) can provide interactive book assets, featuring opening and closing of a book, and animations of story elements or narrated text that appears on top of the open book. The VR space manager 140 can animate the book opening and closing, enhancing engagement.

In some implementations, the system (e.g., VR space manager 140) can support dynamic object scaling (e.g., growing and shrinking an object) through dual-controller input (e.g., input from two joysticks). For example, when both grip buttons of the two joysticks are held and the object is grasped at two designated points, (1) movement of the controllers away from each other can increase the object's scale (e.g., grow the object), and (2) movement toward each other reduce the object's scale (e.g., shrink the object). Scaling limits can be defined per object to ensure appropriate interaction boundaries

In some implementations, the system (e.g., motion tracker 162) can enhance the virtual therapeutic environment through the integration of eye-tracking technology (e g., using HMD or smart glasses) and vibration feedback mechanisms (e.g., vibration motors). This system can be particularly designed to accommodate children who may experience physical disabilities or who are confined to bed due to illness, and who may therefore be unable or prefer not to use hand-based controllers. The integration of eye-tracking can allow users to interact with the virtual environment through ocular movement. Devices supporting eye-tracking technology can enable the system to detect and interpret movement and gaze direction of the user's eyes.

In some implementations, the system (e.g., haptic output manager 192) can generate tactile feedback for users who rely on eye-tracking. The system can include vibration motors (e.g., vibration motors 150V) connected to a controller (e.g., an Arduino* microcontroller). For example, these motors can be positioned on the user's forearms, near the wrists preferably, to deliver haptic signals When an object is selected or manipulated through eye movement, the system can communicate with the controller to activate the vibration motors, thereby providing the user with a physical indication that the object has been engaged. In another example, when the system (e.g., VR motion tracker 162, symptom detector 164) detects (e.g., from a motion of a client) a symptom of a mental illness or a severity level thereof, the system (E.g., haptic output manager 192) can control the vibration motor to generate a haptic output corresponding to the severity level of the symptom. In some implementations, the system (e.g., haptic output manager 192) can provide this feedback mechanism to replicate the tactile experience typically associated with handheld VR controllers. The haptic output manager 192 can vary or adjust the intensity of the vibration depending on the nature of the interaction. For example, the intensity of the vibration can be adjusted according to the force implied by the user's gaze when releasing or dropping an object.

In some implementations, the system (e.g., motion tracker 162) can provide an eye-tracking component (e.g., in communication with the client's controller supporting eye-tracking technology) to monitor the client's gaze and correlate specific eye movements with object selection and manipulation. This can allow users to pick up and control virtual objects using only their eyes, thereby expanding accessibility for users with limited motor function or users who have disabilities or are bedridden due to illness.

In some implementations, the vibration motors can be compact and connected to a controller that may be placed on a surface near the vibration motors. The wiring between the motors and the controller can be sufficiently long to accommodate various physical arrangements. The controller can operate independently of (e.g., without connecting and/or communicating with) a computer, as its firmware is preloaded onto the device.

In addition to providing feedback to the user (e.g., client, child, patient), the vibration motors can be affixed to the therapist's arms. This configuration can allow the therapist to receive real-time haptic cues corresponding to the client's actions within the virtual environment. Such feedback may enhance the therapist's attunement to the client's emotional state and behavioral intensity. For example, a forceful interaction by the client may result in a stronger vibration, prompting the therapist to explore the underlying emotional context. The system (e.g., display manager 184) can cast the visual and audio output of the HWD of the child onto a display or a VR application to observe the child's behaviors/motions in the virtual space. This dual-sensory modality (e.g., visual awareness and tactile cues) parallels the experience of observing a child physically throwing a toy, with the added dimension of tactile perception. The therapist can maintain visual awareness of the client's activity through the casting feature available on VR devices, thereby ensuring that the therapist can observe and interpret the client's behavior within the virtual space.

In some implementations, the system can enable conducting a virtual play therapy through the use of a 3D digital playroom accessible via web or mobile platforms. To begin the process of conducting the virtual play therapy, the system (e.g., user interface manager 130) can allow both the therapist and the client to create individual accounts on a designated website. The user interface manager 130 can provide the therapist with access to a practice interface, allowing unrestricted exploration of the virtual environment prior to initiating a session. When ready to begin a session, the system can generate a unique access code for the therapist. To begin the session, the therapist and client may connect through any preferred videoconferencing platform, such as Zoom. The therapist can transmit the session code to the client via the conferencing platform or through email. Upon receiving the code, the client can be prompted to enter the code into the system, thereby gaining access to the virtual playroom. The client may share their screen through the conferencing platform or by casting the client's HWD on a display or a VR application that the therapist can watch. In this manner, the system can enable the therapist to observe the session in real time. The code may be time-limited and may expire after a predetermined duration to ensure that the virtual environment is used exclusively for therapeutic purposes. In some implementations, the system can use the voice/video chat manager 182 instead of the external videoconferencing platform to streamline this process

In some implementations, the system can provide a virtual playroom as a digitally rendered three-dimensional space that replicates the physical characteristics of a real-world playroom, including architectural features such as walls, windows, and/or doors. The virtual playroom can contain a variety of 3D furniture and interactive toys that can be grabbed, moved, dropped, and/or manipulated. For example, the client can use their keyboard to move a camera (e.g., perspective or view of the player) around the playroom. In some implementations, the camera can have two hands/controllers attached thereto, and the client (e.g., child) can move the hands and use the hands to manipulate the objects. These hands allow the client to interact with objects in the environment. Many of the objects include additional sensory features, such as sound effects or visual animations, to enhance realism and engagement. The virtual playroom serves as the medium through which therapeutic sessions are conducted.

In some implementations, the system (e.g., VR space manager 140) can build/design/provide a virtual environment in which the child and the therapist interact in the same space, so that the virtual space is comfortable for the child, as well as conducive for the therapist to carry out their practice. In synergetic play therapy, the therapist can be the most important tool/toy for identifying emotions, and the child can bring the therapist into their world. The therapist can then attune themselves to the child to identify specific emotions or themes present in their development. Based on this information, the VR space manager 140 can provide different areas in the virtual room to represent different types of play, such as a baby section and kitchen for nurturing themes, a playpen for themes of aggression, and a city to emulate real-life environments, etc.

In some implementations, the system does not retain session data. Only the therapist, and potentially the client's family, can have visibility into the client's activity. For improved security, the programs (for one or more of the user interface manager 130, the VR space manager 140, the VR motion analyzer 160, the user communication manager 180, and/or the notification manager 190) can be hosted on a secure server and not be hosted on a cloud-based server. Access to the server and the application can be restricted exclusively to the play therapy website.

In some implementations, the system (e.g., VR space manager 140) can provide several interactive elements within the environment which are programmed using a programming language such as C #. The VR space manager 140 can model most toys after real-world counterparts, while other toys incorporate features that are uniquely enabled by digital technology. For instance, a virtual frying pan may emit sizzling sounds and visual steam effects, enhancing the realism of the play experience and expanding the child's expressive capacity. Some toys include particle systems that simulate physical phenomena, such as a watering can that releases water particles when tilted. These effects may be either user-controlled or automated, depending on the object.

In some implementations, the system (e.g., user interface manager 130) can provide an XR simulator in a virtual game platform (e.g., Unity®). The simulator can include a set of keyboard and mouse controls. Users can look around using mouse movement, navigate the space using the W, A, S, and D keys, for example, and/or adjust vertical positioning with the E and Q keys, for example. The system can support additional commands including resetting the avatar's position, grabbing and dropping objects, throwing items, cleaning the room, and triggering sound effects, for example.

In some implementations, the system (e.g., user interface manager 130) can provide a VR version which can utilize controllers (e.g., two joysticks) and button inputs of the controllers. For example, the left joystick can enable movement, while the right joystick allows for rotation or teleportation. Each of the joysticks or controllers may have a primary button, a secondary button, and/or trigger button. The primary and secondary buttons on both controllers (e.g. both joysticks) can adjust vertical positioning. Gripping actions on joysticks can be used to grab, drop, or throw objects, and the trigger buttons can activate sound effects.

In some implementations, the system (e.g., VR motion analyzer 160) can provide a virtual reality-based therapeutic environment in which a therapist observes and engages with a client, typically a child, as they navigate a three-dimensional playroom. Within this environment, the therapist may apply standard play therapy or psychotherapy techniques while monitoring a range of behavioral and spatial interactions.

In some implementations, the system (e.g., VR motion analyzer 160) can enable the therapist to monitor and analyze the client's behaviors, movements, and/or expressions within the virtual environment. This analysis can support the diagnosis and treatment of mental health conditions, the resolution of behavioral challenges, and the achievement of therapeutic goals established by the client or their family.

In some implementations, the system (e.g., VR motion tracker 162) can enable the tracking of how a client interacts with virtual objects, including the manner in which toys are thrown or struck against the ground. Upon the release of an object, the VR motion tracker 162 can record its position in 3D space by storing the x, y, and z coordinates in separate variables. The VR motion tracker 162 can then track the movement of the controller over a defined time interval, such as five seconds, and calculate the velocity by dividing the change in position by the elapsed time.

In some implementations, the system (e.g., VR motion tracker 162) can monitor client interaction with their virtual body representation, particularly the hands. For example, the VR motion tracker 162 can perform body tracking by continuously recording the spatial coordinates of the HMD and the handheld controllers. In some implementations, the system (e.g., VR space manager 140) can divide the virtual environment into distinct zones, each with its own boundary and timer. When the HMD of the client enters a designated zone, the timer of that zone can initiate, and when the HMD exits, the timer can pause. At the conclusion of a session, when the virtual playroom is closed, the system (e.g., notification manager 190) can generate a report detailing the duration spent in each zone, which is then made available to the therapist.

In some implementations, the system (e.g., VR motion tracker 162) can track object preferences by recording which items the client selects or gravitates toward. Each object may include a script that increments a counter every time it is selected, allowing for cumulative analysis of object interaction frequency.

In some implementations, the system (e.g., VR motion tracker 162) can monitor vertical movement within the environment to assess the client's intent. For example, upward or downward movement may indicate attempts to reach an object, simulate flight, or seek concealment below ground level. The system can record the client's position (e.g., height) relative to the floor and ceiling to analyze these behaviors.

In some implementations, the system (e.g., VR motion tracker 162) can track auditory interaction. When a sound is triggered by a trigger button of the client's controller, a timer can begin and run for approximately twenty seconds. During this interval, the system can count the number of times the trigger button is pressed, allowing for an analysis of sound-related engagement frequency.

In some implementations, the system (e.g., VR motion tracker 162) can track/record/monitor the time required for a client to pick up an object. The system can also track/record/monitor the time required for a client to drop an object. The system can compare the height of the object at the moment of release with the height of the floor to determine whether the client attempts to place the object gently or allows it to fall from a greater height.

In some implementations, the system (e.g., VR motion tracker 162) can analyze spatial boundaries by observing how the client approaches the virtual walls and ceiling The system can determine whether the client remains within the confines of the room or attempts to move beyond its limits, by comparing the client's position with the x and z coordinates of the room's structural boundaries.

In some implementations, the system can enable a therapeutic process conducted within a virtual environment, wherein the therapist engages the client through a series of interactions. The process may involve the introduction of prompts or situational narratives designed to guide the client's play and elicit expressive behavior. These scenarios can serve as a foundation for therapeutic dialogue and emotional exploration. Throughout the session, the therapist may initiate conversations with the client regarding specific experiences or topics relevant to the client's psychological healing. The therapist can provide emotional support during moments of distress, including episodes of emotional breakdown or the emergence of difficult affective states.

In some implementations, the system (e.g., VR space manager 140) can provide the virtual playroom and its interactive elements that can serve as a medium for therapeutic engagement. Toys and objects within the environment may be used as symbolic tools to initiate conversation and facilitate emotional expression. The therapist can remain actively engaged with the client during play, interpreting behaviors and guiding the session in accordance with established therapeutic principles.

In some implementations, the system (e.g., symptom detector 164, notification manager 190, haptic output manager 192) can support the diagnosis and treatment of a wide range of mental health conditions and emotional challenges. These may include, but are not limited to, attention-deficit/hyperactivity disorder (ADHD), Autism spectrum disorders, personality disorders, bipolar disorder, anxiety, depression, schizophrenia, conduct disorders, and social disorders. The process can also address the psychological effects of trauma, obsessive-compulsive disorder, persistent obsessions and compulsions, and behaviors that may be characterized as malicious, inappropriate, or dangerous.

Additionally, the method using the virtual spaces may be applied in cases involving psychological regression or the manifestation of defense mechanisms, as well as in the treatment of phobias, fears, and the emotional consequences of neglect or abuse. The therapeutic framework can be adaptable to any practice, technique, or method commonly employed in psychotherapy or play therapy, and can be designed to support individualized treatment goals established by the therapist in collaboration with the client and, where appropriate, the client's family.

In some implementations, the system (e.g., system 100) can be used for diagnosing one or more mental illnesses (e.g., ADHD, Autism, personality disorders, bipolar disorder, anxiety, depression, schizophrenia, conduct disorders, social disorders, etc.) using a VR environment in which a first user (e.g., client) and a second user (e.g., therapist) are present. The system may receive, from a head worn device (HWD) of the first user (e.g., HMD 150H), first data representing a first motion of the first user in the VR environment. The system (e.g., symptom detector 164) may determine, based on the first data (and using the data stored in the databases 120), that the first motion of the first user (e.g., releasing, throwing, or hitting one or more objects) relates to a first symptom of the one or more mental illnesses (e.g., symptoms of conduct disorders). The system may receive, from the HWD of the first user, second data representing at least one of an intensity, a boundary, or a frequency of the first motion of the first user (e.g., the intensity of releasing, throwing, or hitting one or more objects which can be detected by the VR motion tracker). The system (e.g., symptom detector 164) may determine, based on the second data, a severity level of the first symptom (e.g., severity level of symptoms of conduct disorders). The system (e.g., haptic output manager 192) may control a wearable device of the second user (e.g., vibration motor worn by the therapist) to generate a first haptic output corresponding to the severity level of the first symptom (e.g., vibration that has intensity corresponding to the severity level of symptoms of conduct disorders).

In some implementations, the VR environment may include a plurality of virtual spaces (e.g., virtual playrooms) in each of which the first user (e.g., client) selects and/or manipulates one or more objects (e.g., toys) during a play therapy session. In some implementations, the system (e.g., display manager 184) may be further configured to cast from the HWD of the first user to a display device of the second user. In some implementations, the intensity of the first motion of the first user may represent an intensity of the first user releasing, throwing, or hitting one or more objects in the VR environment. In determining the severity level of the first symptom, the system (e.g., VR motion tracker 162) may be configured to determine the severity level of the first symptom based at least on a gaze direction or a movement pattern of an eye of the first user while the user releases, throws, or hit the one or more objects in the VR environment.

In some implementations, the boundary of the first motion of the first user may represent a boundary of the first motion of the first user with respect to a location of at least one of a floor, a ceiling, or a wall of the VR environment. In some implementations, the frequency of the first motion of the first user may represent a number of repeating the first motion of the first user within a predetermined time (e.g., repeating playing sounds within 20 seconds).

In some implementations, the system may be configured to receive, from the HWD of the first user, third data representing one or more objects (e.g., toys) that relate to a second motion of the first user in the VR environment. The system (e.g., VR motion tracker 162) may be configured to determine, based on the third data, that the second motion of the first user relates to a second symptom of the one or more mental illnesses (e.g., repeatedly picking up or avoiding a certain toy during the play therapy might be a symptom of past trauma). The one or more processors may be configured to determine, based on the third data, a severity level of the second symptom (e.g., how often the certain toy is picked up or avoided which can be detected by VR motion tracker 162). The system (e.g., haptic output manager 192) may be configured to control the wearable device of the second user (e.g., vibration motor worn by the therapist) to generate a second haptic output corresponding to the severity level of the second symptom (e.g., vibration that has intensity corresponding to the severity level of symptoms of the past trauma). The third data may represent that the first user picks up or gravitates towards the one or more objects in the VR environment. In determining the severity level of the second symptom, the system (e.g., symptom detector 164) may be configured to determine, based on the number of the one or more objects picked up or gravitated towards by the first user, the severity level of the second symptom.

Various implementations in the present disclosure have one or more of the following advantages and benefits.

First, implementations in the present disclosure can provide a fully immersive, 3D virtual playroom that replicates real-world play environments. Some implementations in the present disclosure can allow clients (e.g., children) to move freely, grab, manipulate, and interact with toys in a way that mimics physical play, unlike 2D virtual playrooms.

Second, implementations in the present disclosure can enable users (e.g., therapists) to join the virtual space, observe, and interact with the client (e.g., child) in real time. Some implementations in the present disclosure can support a voice chat, a video chat, and/or avatar-based presence, fostering a sense of co-presence and emotional attunement.

Third, implementations in the present disclosure can track detailed behavioral metrics such as object interaction, movement patterns, time spent in specific zones, and/or emotional responses. Some implementations in the present disclosure can generate various forms of feedback (e.g., haptic feedback indicating the symptom level of a detected symptom or session reports that can support diagnosis and treatment planning).

FIG. 2 is a block diagram illustrating an example of a computing system according to some implementations.

Referring to FIG. 2, the illustrated example computing system 200 includes one or more processors 210 in communication, via a communication system 240 (e.g., bus), with memory 260, at least one network interface controller 230 with network interface port for connection to a network (not shown), and other components, e.g., an input/output (“I/O”) components interface 250 connecting to a display (not illustrated) and an input device (not illustrated). Generally, the processor(s) 210 will execute instructions (or computer programs) received from memory. The processor(s) 210 illustrated incorporate, or are directly connected to, cache memory 220. In some instances, instructions are read from memory 260 into the cache memory 220 and executed by the processor(s) 210 from the cache memory 220.

In more detail, the processor(s) 210 may be any logic circuitry that processes instructions, e.g., instructions fetched from the memory 260 or cache 220. In some implementations, the processor(s) 210 are microprocessor units or special purpose processors. The computing device 200 may be based on any processor, or set of processors, capable of operating as described herein. The processor(s) 210 may be single core or multi-core processor(s). The processor(s) 210 may be multiple distinct processors.

The memory 260 may be any device suitable for storing computer readable data. The memory 260 may be a device with fixed storage or a device for reading removable storage media. Examples include all forms of non-volatile memory, media and memory devices, semiconductor memory devices (e.g., EPROM, EEPROM, SDRAM, and flash memory devices), magnetic disks, magneto optical disks, and optical discs (e.g., CD ROM, DVD-ROM, or Blu-Ray® discs). A computing system 200 may have any number of memory devices as the memory 260.

The cache memory 220 is generally a form of computer memory placed in close proximity to the processor(s) 210 for fast read times. In some implementations, the cache memory 220 is part of, or on the same chip as, the processor(s) 210. In some implementations, there are multiple levels of cache 220, e.g., L2 and L3 cache layers.

The network interface controller 230 manages data exchanges via the network interface (sometimes referred to as network interface ports). The network interface controller 230 handles the physical and data link layers of the OSI model for network communication. In some implementations, some of the network interface controller's tasks are handled by one or more of the processor(s) 210. In some implementations, the network interface controller 230 is part of a processor 210. In some implementations, a computing system 200 has multiple network interfaces controlled by a single controller 230. In some implementations, a computing system 200 has multiple network interface controllers 230. In some implementations, each network interface is a connection point for a physical network link (e.g., a cat-5 Ethernet link). In some implementations, the network interface controller 230 supports wireless network connections and an interface port is a wireless (e.g., radio) receiver/transmitter (e.g., for any of the IEEE 802.11 protocols, near field communication “NFC”, Bluetooth, ANT, or any other wireless protocol). In some implementations, the network interface controller 230 implements one or more network protocols such as Ethernet. Generally, a computing device 200 exchanges data with other computing devices via physical or wireless links through a network interface. The network interface may link directly to another device or to another device via an intermediary device, e.g., a network device such as a hub, a bridge, a switch, or a router, connecting the computing device 200 to a data network such as the Internet.

The computing system 200 may include, or provide interfaces for, one or more input or output (“I/O”) devices 250. Input devices include, without limitation, keyboards, microphones, touch screens, foot pedals, sensors, MIDI devices, and pointing devices such as a mouse or trackball. Output devices include, without limitation, video displays, speakers, refreshable Braille terminal, lights, MIDI devices, and 2-D or 3-D printers.

Other components may include an I/O interface, external serial device ports, and any additional co-processors. For example, a computing system 200 may include an interface (e.g., a universal serial bus (USB) interface) for connecting input devices, output devices, or additional memory devices (e.g., portable flash drive or external media drive). In some implementations, a computing device 200 includes an additional device such as a co-processor, e.g., a math co-processor can assist the processor 210 with high precision or complex calculations.

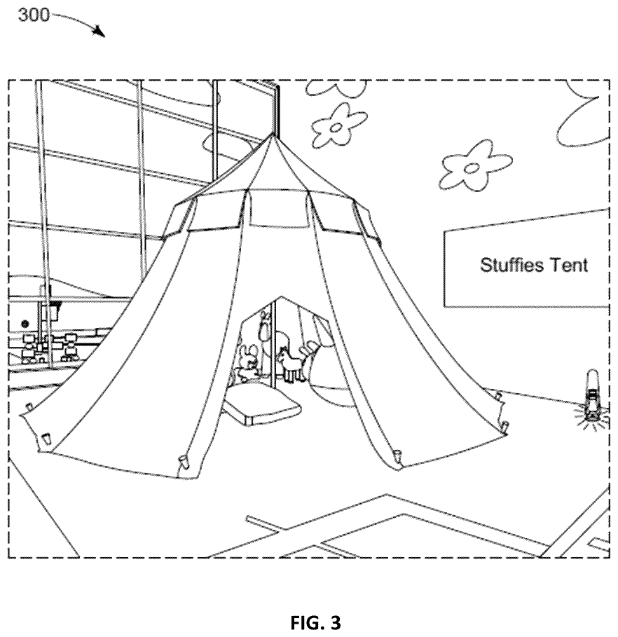

FIG. 3 to FIG. 14 are example virtual spaces (e.g., virtual playrooms or different sections in a virtual playroom) 300, 400, 500, 600, 700, 800, 900, 1000, 1100, 1200, 1300, 1400 for diagnosing one or more mental illnesses, according to some implementations. These spaces may not be all separate playrooms; they may just be sections of one playroom. The virtual playroom described in the present disclosure can be developed using a game engine (e.g., Unity® engine) and can incorporate a variety of assets sourced from a game asset store (e.g., Unity® Asset Store). The virtual environment can be divided into multiple thematic sections, each designed to support specific therapeutic objectives aligned with established principles of play therapy. These themes may include, but are not limited to, nurturing, aggression, and real-world simulation, and are represented through curated collections of interactive objects and spatial arrangements.

FIG. 3 is an example virtual playroom (e.g., a virtual stuffed animal tent) in a virtual play therapy platform, according to some implementations. For example, the tent is furnished with 9 stuffed animals and 3 pillows.

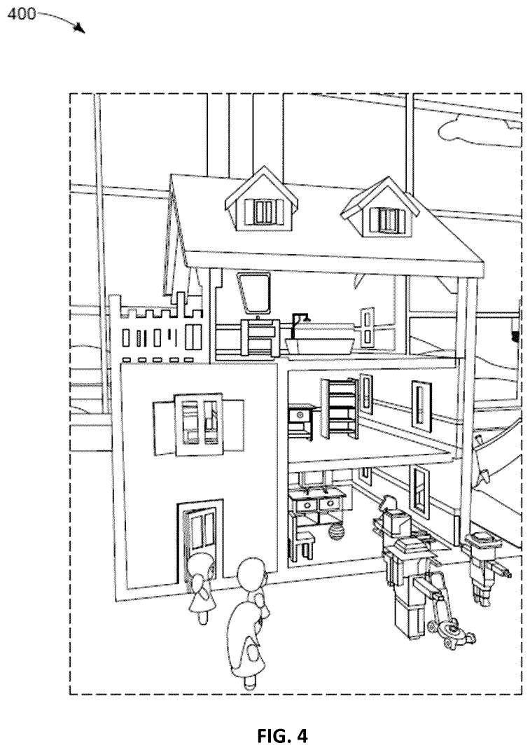

FIG. 4 is an example virtual playroom (e.g., a virtual dollhouse section) in a virtual play therapy platform, according to some implementations. For example, the dollhouse is accompanied by 6 dollhouse toys and 7 dolls and miniature furnishings. The miniature furnishings are designed to model home life or interactions between family members.

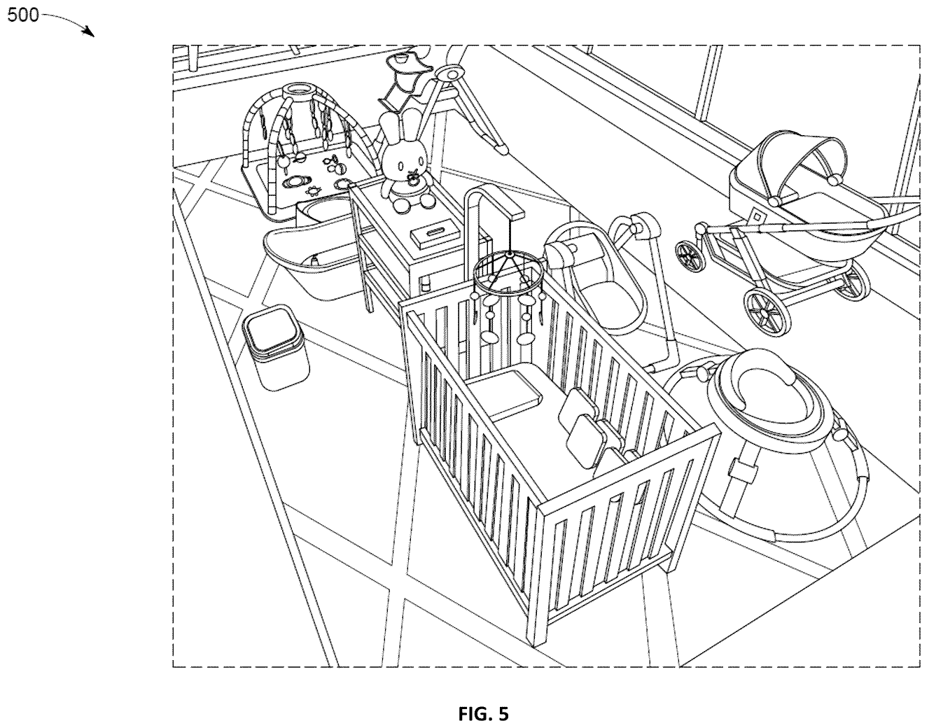

FIG. 5 is an example virtual playroom (e.g., a virtual nurturing section or baby care section) in a virtual play therapy platform, according to some implementations. For example, a baby care section can be equipped with a baby bunny, 1 crib and pillow, 1 brush, 1 comb, 1 thermometer, 1 towel, 2 diapers, 2 pacifiers, 1 bottle, 2 creams, 1 baby powder, 7 baby toys, 1 play mat, 1 wet wipes box, 2 stuffed animals, 5 baby pillows, 1 bathtub, 1 rubber duck, 1 shampoo, 1 stroller, 1 swing, 1 baby bouncer, 1 potty seat, 1 highchair, 1 baby bowl, and 1 baby spoon. These elements can be used to evoke caregiving behaviors and facilitate emotional expression related to attachment and dependency.

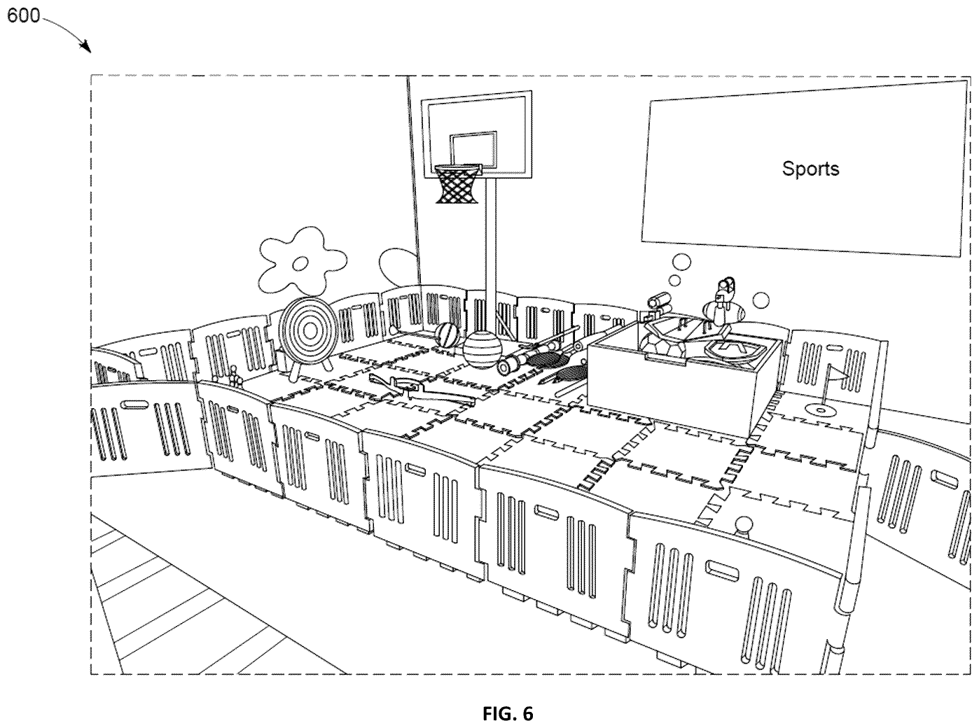

FIG. 6 is an example virtual playroom (e.g., a virtual sports pen) in a virtual play therapy platform, according to some implementations. For example, the sports section features a variety of athletic equipment, including 1 basketball, basketball hoop, 1 soccer ball, 1 football, 3 tennis rackets, 2 tennis balls, 1 baseball bat, 1 baseball, 2 swords, 3 shields, a bowling ball and pins, 1 golf club, 1 golf ball, 1 golf hole, 1 frisbee, 1 bouncy ball, 1 bow and suction arrow, 1 target, 1 bazooka, 1 boomerang, and 1 lightsaber. This area supports the exploration of themes related to competition, physicality, and assertiveness.

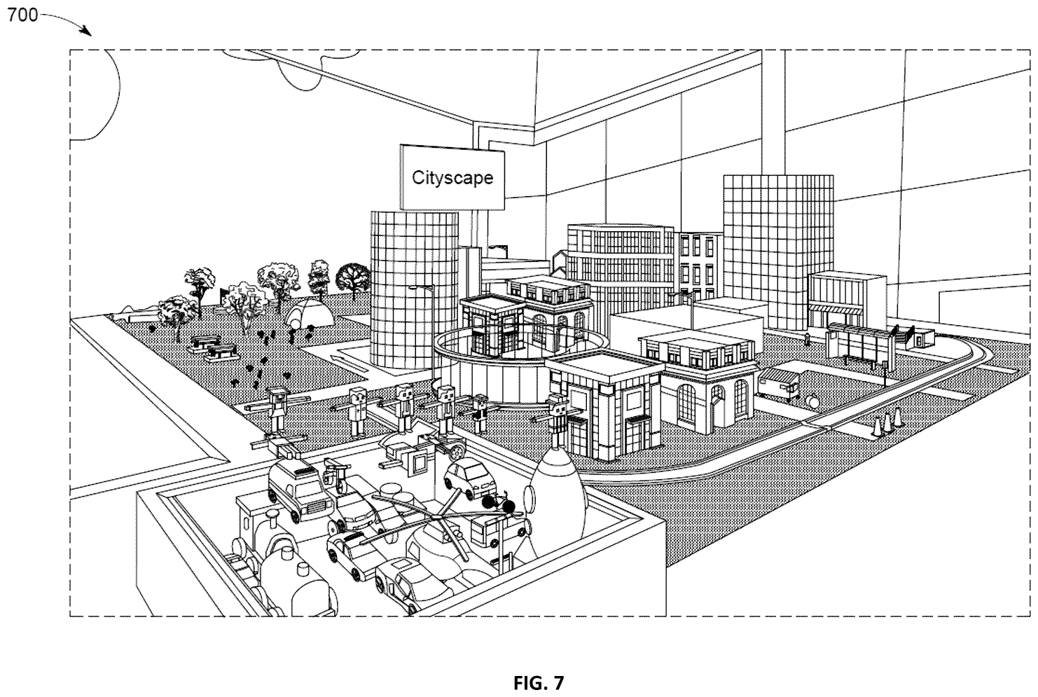

FIG. 7 is an example virtual playroom (e.g., a virtual cityscape) in a virtual play therapy platform, according to some implementations. The cityscape section can replicate an urban environment with buildings, vehicles, figures, and recreational equipment. For example, the virtual cityscape includes 20 city buildings, 4 cars, 1 police car, 1 ambulance, 1 dump truck, 10 figures, 1 taxi, 1 city playground, 4 trains, 1 helicopter, 1 rocket ship, 1 bicycle, 1 tricycle, 1 pogo stick, 2 roller skates, 1 skateboard, 1 scooter, 1 go-kart, and 1 toddler bike, offering opportunities for role-play and the enactment of real-life scenarios.

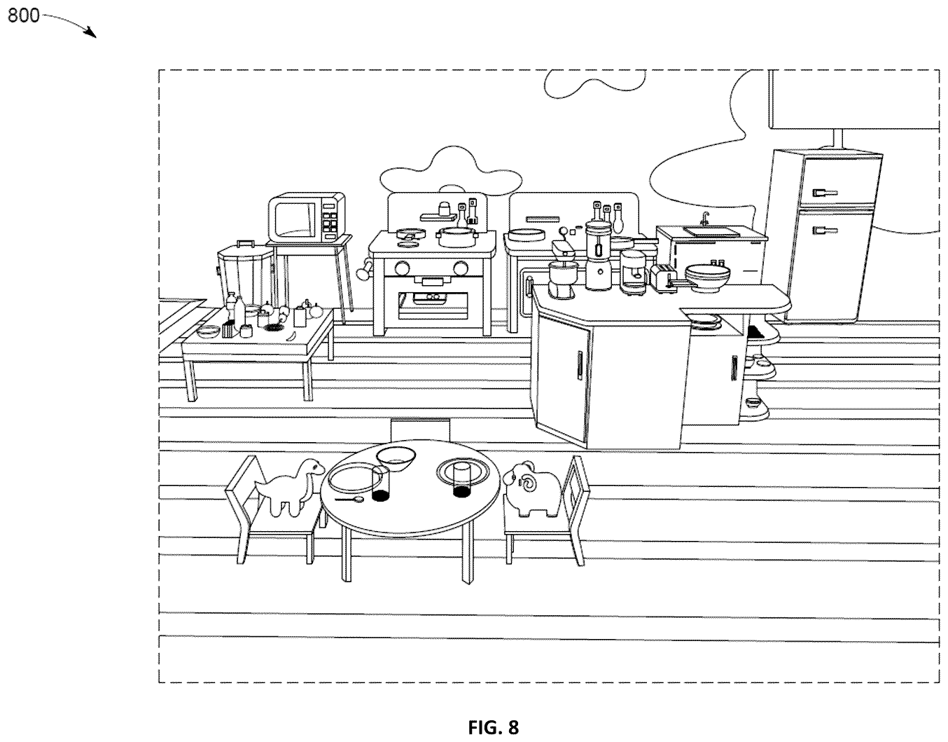

FIG. 8 is an example virtual playroom (e.g., a virtual kitchen) in a virtual play therapy platform, according to some implementations. For example, the kitchen area is equipped with 35 foods, 4 spices, counter, 1 stove, table and chairs, trash can, 4 pots, 2 pans, 5 spoons, 4 forks, 3 knives, 2 cutting boards, 2 plates, 4 bowls, 3 glasses, 2 cups and saucers, 1 teakettle, 1 coffee machine, 1 electric mixer, 1 blender, 1 toaster, 1 microwave, 1 fridge, 1 kitchen sink, 1 cooking tray, and 1 piece of trash, enabling the simulation of domestic routines and the expression of nurturing or social behaviors.

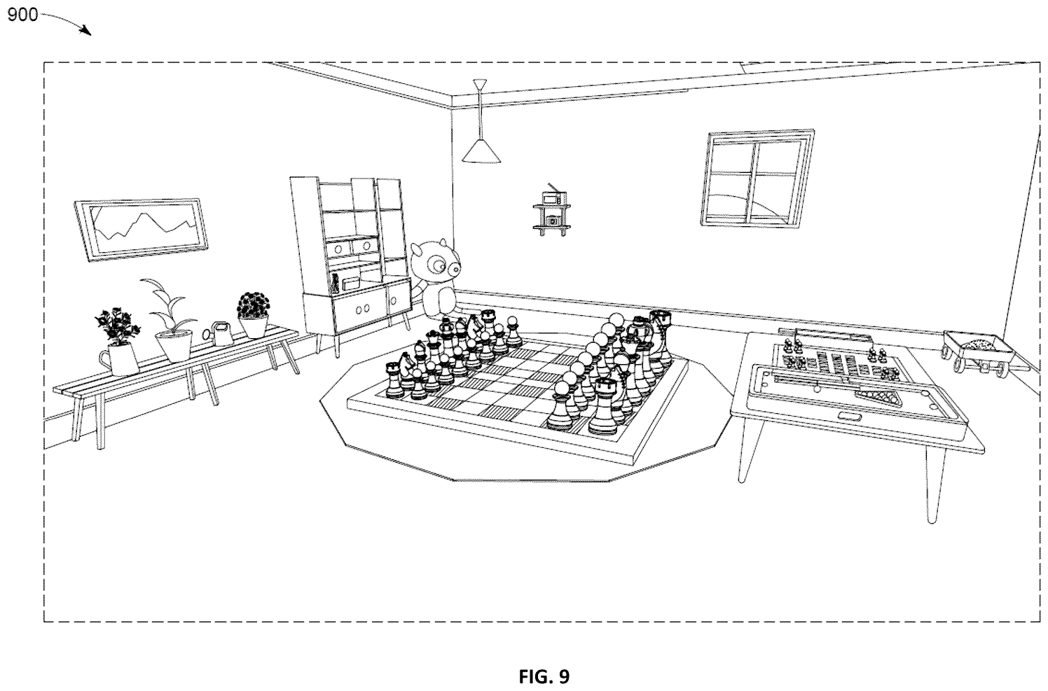

FIG. 9 is an example virtual playroom (e.g., a virtual game room) in a virtual play therapy platform, according to some implementations. For example, the virtual game room includes 1 chess board and pieces, 1 “Sorry” game, 1 box of dominoes, 1 pool table with balls and cues, and 1 dartboard and darts.

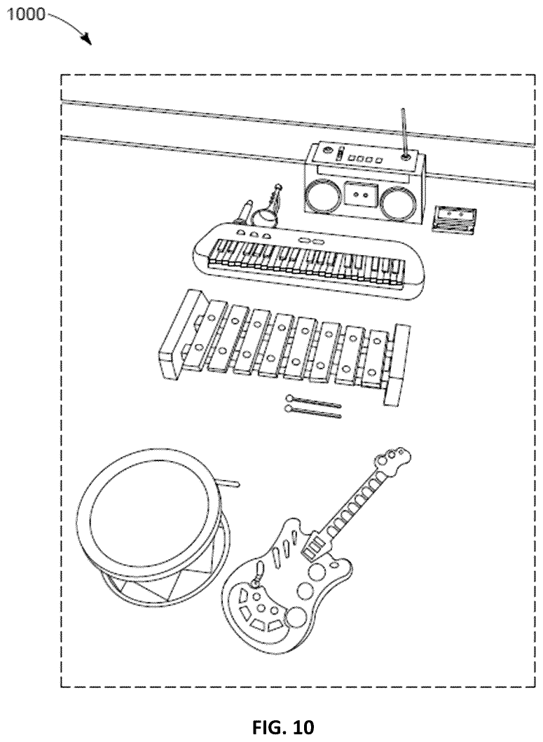

FIG. 10 is an example virtual playroom (e.g., a virtual music section) in a virtual play therapy platform, according to some implementations. For example, the virtual music section includes 1 piano, 1 trumpet, 1 guitar, 1 xylophone, 1 microphone, 1 drum and stick, and 1 cassette player and 3 tapes.

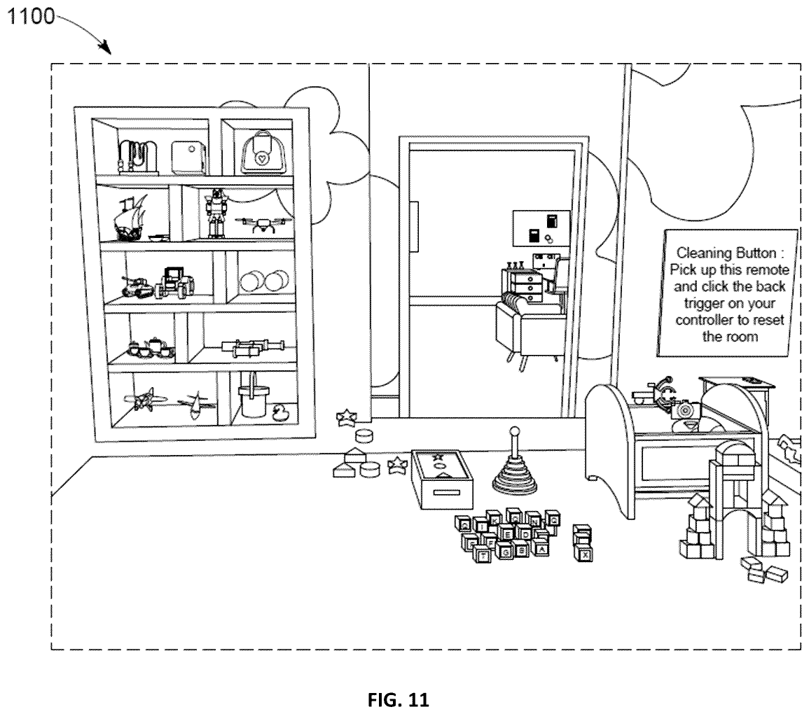

FIG. 11 is an example virtual playroom (e.g., a virtual general toys section) in a virtual play therapy platform, according to some implementations. The virtual general toys section can contain a broad assortment of play objects. For example, the virtual general toys section includes wooden blocks, alphabet blocks, 1 bucket, 1 shovel, 1 toy stacker, 1 shape puzzle, 1 dice, 1 purse, 1 tea set, 3 boats, 1 toy tractor, 1 toy tank, 1 drone, 1 robot, 2 planes, 1 telescope, 1 pair of binoculars, 1 beads toy, 1 toy camera, 1 magic want, 1 mirror, and 1 toy phone.

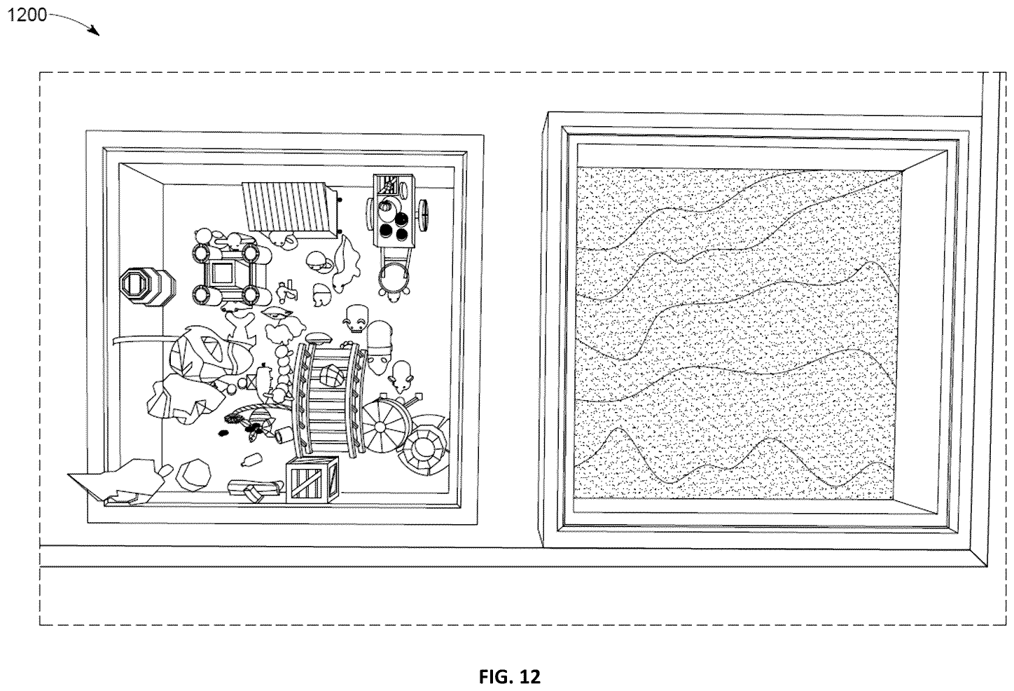

FIG. 12 is an example virtual playroom (e.g., a virtual sand tray section) in a virtual play therapy platform, according to some implementations. The sand tray area can include a sand container and a large collection of figures, supporting symbolic play and narrative construction. For example, the virtual sand tray includes 1 sand tray, 74 figures, etc.

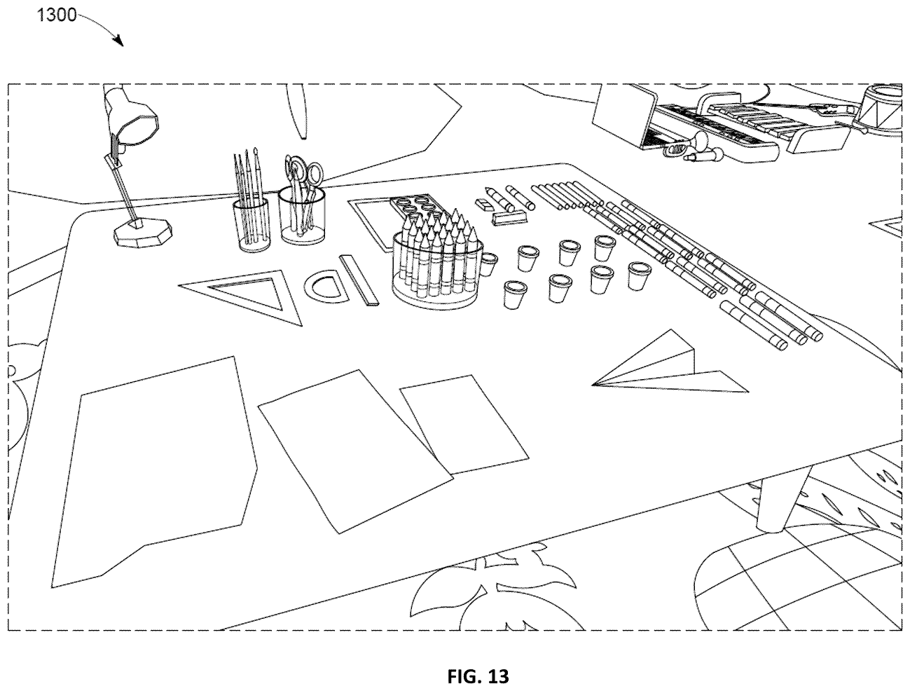

FIG. 13 is an example virtual playroom (e.g., a virtual art table section) in a virtual play therapy platform, according to some implementations. The art table can be stocked with a comprehensive set of creative tools, including markers, paints, pencils, brushes, and other drawing instruments, allowing for expressive and projective activities. For example, the virtual art table includes 3 papers, 1 paper airplane, 16 markers, 8 paints, 1 watercolor, 7 colored pencils, 1 pen, 1 highlighter, 1 eraser, 4 pencils, 2 scissors, 4 paintbrushes, 1 ruler, 2 protractors, 20 crayons, etc.

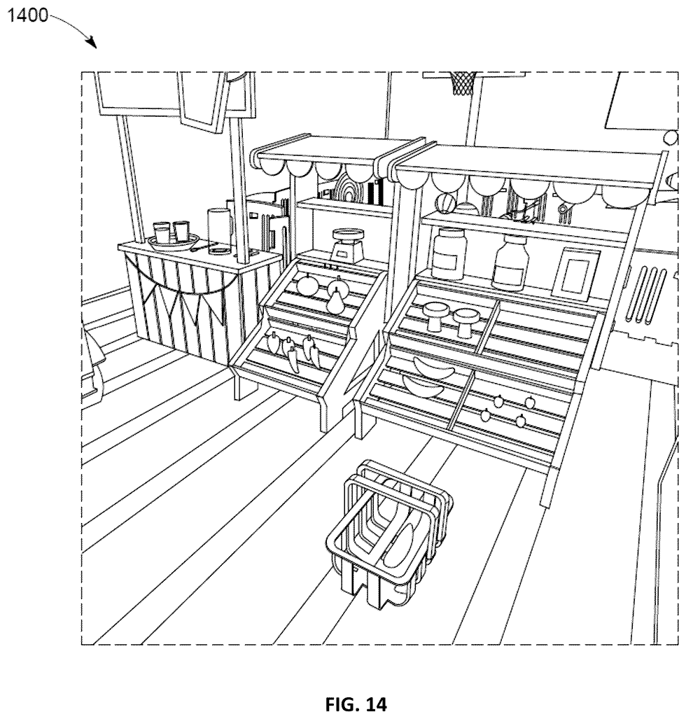

FIG. 14 is an example virtual playroom (e.g., a virtual shopping section) in a virtual play therapy platform, according to some implementations. In some implementations, the virtual shopping section can simulate a market environment with stands, food items, utensils, containers, and a cash register, facilitating role-play related to commerce and social interaction. For example, the virtual shopping section includes 2 shopping stands, a lemonade stand, 2 spoons, 2 glasses, 1 jug, 1 knife, 1 straw, 1 sugar container, 18 foods, 2 jars, 1 box, 1 scale, 1 cash register and money, 1 shopping basket, etc. In some implementations, the system can provide other toys (not shown) such as 1 lawn mower, 1 chair, 1 campfire, 1 umbrella, 1 ball, 1 horseshoe and pike, 1 hula hoop, 1 gravestone, 1 flower, 1 pool, 1 playhouse, 1 chest, 1 slide, 1 tent, more pillows, and more stuffed animals. In some implementations, the system can provide additional decorative and functional elements (not shown) including, for example, outdoor sports fields; a bathroom with 1 bathtub and shower, 1 stuffed animal, 1 rubber duck, 1 shampoo, 1 conditioner, 1 bar of soap, 5 towels, 1 comb, 1 hairbrush, 1 thermometer, 1 hand soap bottle, 1 toothbrush, 1 toothpaste tube, 1 sink, 1 trash can, 1 toilet, 1 scale, 6 ointments, 1 lotion, 4 creams, 2 bottles, 1 washing machine, 1 dryer, 8 clothes, and 1 hairdryer; and a relaxation area with 2 sofas, 1 coffee table, 1 wardrobe, 1 bed, 1 vanity table, 1 desk, 1 office chair, 1 TV and remote, 1 video game controller, 2 pillows, 11 stuffies, 1 alarm clock, 3 balloon animals, 1 calendar, 7 books, 3 lamps, 5 hats, 1 wipe off board and marker, 1 laptop, 1 tablet, 1 calculator, 1 stapler, 1 tape dispenser, 2 beakers, 1 vial, 1 petri dish, 1 dropper, 1 camera, 1 radio, 4 plants, 1 watering can, 1 wagon, 1 pinwheel, 1 abacus, 1 newton's balls, 1 bubbles, 1 microscope, and 1 piggy bank. In some implementations, the system can provide expanded furnishings, medical equipment, and hospital-themed toys (not shown) to support therapeutic work in clinical and institutional settings. For example, a medical play section with 12 animals, 7 pet beds, 4 pillows, 1 scale, 1 lab table, 1 incubator, 1 exam table, 1 blood pressure cuff, 1 stethoscope, 1 insulin tracker and sticker, 1 pulse oximeter, 1 thermometer, 4 pills, 1 pill bottle, 2 pill packets, 1 vaccine, 4 pet foods, and 1 clipboard; a hospital room with 1 doctor, 1 nurse, 2 chairs, 2 hospital beds, 2 nightstands, 2 rolling tables, 2 IVs, 1 ventilator machine, 1 wheelchair, 1 ultrasound machine, and 1 stuffed animal; and an operating room with 1 operating table, operating lights, 1 blood IV, 1 scale, 1 anesthesia machine, 1 endoscopy machine, 1 heart monitor, 1 kidney tray, 1 x-ray machine and an x-ray. 1 sink, 3 covers, 9 surgery tools, 1 dialysis machine, 1 apheresis machine, 1 MRI machine, 1 radio scanner, 1 ambulance and transport bed and 1 doctor.

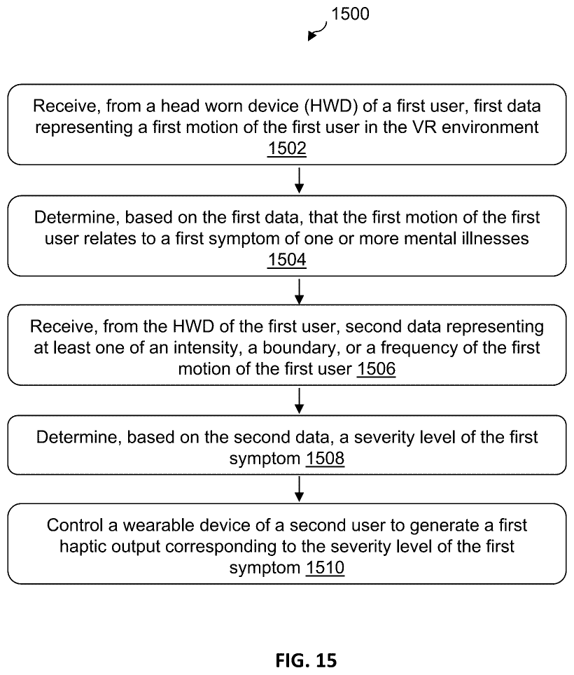

FIG. 15 is a flowchart illustrating an example methodology/process 1500 for diagnosing one or more mental illnesses using a VR environment, according to some implementations. In some implementations, the process 1500 is performed by one or more processors (e.g., processor 210), a device (e.g., a user device 150-1, 150-2, . . . , 150-n, a HWD 150H, a smart glasses 150S, a vibration motor 150V, and/or one or more processors 210 thereof) or a system (e.g., a VR mental illness diagnosis system 100 and/or one or more processors 210 thereof). In some embodiments, the process 1500 is performed by other entities. In some embodiments, the process 1500 includes more, fewer, or different steps than shown in FIG. 15.

In some implementations, the one or more processors, the device, or the system can diagnose one or more mental illnesses (e.g., ADHD, Autism, personality disorders, bipolar disorder, anxiety, depression, schizophrenia, conduct disorders, social disorders, etc.) using a virtual reality (VR) environment (e.g., virtual therapy platform) in which a first user (e.g., client, patient, child, etc.) and a second user (e.g., play therapist, psychiatrist, teacher, etc.) are present. In some implementations, the VR environment may include a plurality of virtual spaces (e.g., virtual playrooms) in each of which the first user (e.g., client) selects and/or manipulates one or more objects (e.g., toys) during a play therapy session.

In this example methodology, a process 1500 begins at step 1502 by receiving, by the one or more processors (e.g., processor 210 of the VR mental illness diagnosis system 100), from a head worn device (HWD) of the first user (e.g., HMD 150H), first data representing a first motion of the first user in the VR environment.

At step 1504, in some implementations, the one or more processors (e.g., symptom detector 164) may be configured to determine, based on the first data (and using the data stored in the databases 120), that the first motion of the first user (e.g., releasing, throwing, or hitting one or more objects) relates to a first symptom of the one or more mental illnesses (e.g., symptoms of conduct disorders).

At step 1506, in some implementations, the one or more processors may be configured to receive, from the HWD of the first user, second data representing at least one of an intensity, a boundary, or a frequency of the first motion of the first user (e.g., the intensity of releasing, throwing, or hitting one or more objects which can be detected by the VR motion tracker).

In some implementations, the intensity of the first motion of the first user may represent an intensity of the first user releasing, throwing, or hitting one or more objects in the VR environment. In determining the severity level of the first symptom, the one or more processors (e.g., VR motion tracker 162) may be configured to determine the severity level of the first symptom based at least on an gaze direction or a movement pattern of an eye of the first user while the user releases, throws, or hit the one or more objects in the VR environment.

In some implementations, the boundary of the first motion of the first user may represent a boundary of the first motion of the first user with respect to a location of at least one of a floor, a ceiling, or a wall of the VR environment. In some implementations, the frequency of the first motion of the first user may represent a number of repeating the first motion of the first user within a predetermined time (e.g., repeating playing sounds within 20 seconds).

At step 1508, in some implementations, the one or more processors (e.g., symptom detector 164) may be configured to determine, based on the second data, a severity level of the first symptom (e.g., severity level of symptoms of conduct disorders).

At step 1510, in some implementations, the one or more processors (e.g., haptic output manager 192) may be configured to control a wearable device of the second user (e.g., vibration motor worn by the therapist) to generate a first haptic output corresponding to the severity level of the first symptom (e.g., vibration that has intensity corresponding to the severity level of symptoms of conduct disorders).

In some implementations, the one or more processors (e.g., display manager 184) may be further configured to cast from the HWD of the first user to a display device of the second user.

In some implementations, the one or more processors may be configured to receive, from the HWD of the first user, third data representing one or more objects (e.g., toys) that relate to a second motion of the first user in the VR environment. The one or more processors (e.g., VR motion tracker 162) may be configured to determine, based on the third data, that the second motion of the first user relates to a second symptom of the one or more mental illnesses (e.g., repeatedly picking up or avoiding a certain toy during the play therapy might be a symptom of past trauma). The one or more processors may be configured to determine, based on the third data, a severity level of the second symptom (e.g., how often the certain toy is picked up or avoided which can be detected by VR motion tracker 162).

In some implementations, the one or more processors (e.g., haptic output manager 192) may be configured to control the wearable device of the second user (e.g., vibration motor worn by the therapist) to generate a second haptic output corresponding to the severity level of the second symptom. The third data may represent that the first user picks up or gravitates towards the one or more objects in the VR environment. In determining the severity level of the second symptom, the one or more processors (e.g., symptom detector 164) may be configured to determine, based on the number of the one or more objects picked up or gravitated towards by the first user, the severity level of the second symptom.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of blocks in the processes disclosed is an example of illustrative approaches. Based upon design preferences, it is understood that the specific order or hierarchy of blocks in the processes may be rearranged while remaining within the scope of the previous description. The accompanying method claims present elements of the various blocks in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

The various examples illustrated and described are provided merely as examples to illustrate various features of the claims. However, features shown and described with respect to any given example are not necessarily limited to the associated example and may be used or combined with other examples that are shown and described. Further, the claims are not intended to be limited by any one example.

The foregoing method descriptions and the process flow diagrams are provided merely as illustrative examples and are not intended to require or imply that the blocks of various examples must be performed in the order presented. As will be appreciated by one of skill in the art the order of blocks in the foregoing examples may be performed in any order. Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm blocks described in connection with the examples disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The hardware used to implement the various illustrative logics, logical blocks, modules, and circuits described in connection with the examples disclosed herein may be implemented or performed with a general purpose processor, a DSP, an ASIC, an FPGA or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some blocks or methods may be performed by circuitry that is specific to a given function.

In some examples, the functions described may be implemented in hardware, software, firmware, or any combination thereof. If implemented in software, the functions may be stored as one or more instructions or code on a non-transitory computer-readable storage medium or non-transitory processor-readable storage medium. The blocks of a method or algorithm disclosed herein may be embodied in a processor-executable software module which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The preceding description of the disclosed examples is provided to enable any person skilled in the art to make or use the present disclosure. Various modifications to these examples will be readily apparent to those skilled in the art, and the generic principles defined herein may be applied to some examples without departing from the spirit or scope of the disclosure. Thus, the present disclosure is not intended to be limited to the examples shown herein but is to be accorded the widest scope consistent with the following claims and the principles and novel features disclosed herein.

What is claimed is:

1. A device for diagnosing one or more mental illnesses using a virtual reality (VR) environment in which a first user and a second user are present, the device comprising: one or more processors configured to:
receive, from a head worn device (HWD) of the first user, first data representing a first motion of the first user in the VR environment;
determine, based on the first data, that the first motion of the first user relates to a first symptom of the one or more mental illnesses;
receive, from the HWD of the first user, second data representing at least one of an intensity, a boundary, or a frequency of the first motion of the first user;
determine, based on the second data, a severity level of the first symptom; and
control a wearable device of the second user to generate a first haptic output corresponding to the severity level of the first symptom.

2. The device according to claim 1, wherein the VR environment includes a plurality of virtual spaces in each of which the first user selects and/or manipulates one or more objects during a play therapy session.

3. The device according to claim 1, wherein the one or more processors are further configured to:
cast from the HWD of the first user to a display device of the second user.

4. The device according to claim 1, wherein
the intensity of the first motion of the first user represents an intensity of the first user releasing, throwing, or hitting one or more objects in the VR environment.

5. The device according to claim 4, wherein
in determining the severity level of the first symptom, the one or more processors are configured to:
determine the severity level of the first symptom based at least on an gaze direction or a movement pattern of an eye of the first user while the user releases, throws, or hit the one or more objects in the VR environment.

6. The device according to claim 1, wherein
the boundary of the first motion of the first user represents a boundary of the first motion of the first user with respect to a location of at least one of a floor, a ceiling, or a wall of the VR environment.

7. The device according to claim 1, wherein
the frequency of the first motion of the first user represents a number of repeating the first motion of the first user within a predetermined time.

8. The device according to claim 1, wherein the one or more processors are further configured to:
receive, from the HWD of the first user, third data representing one or more objects that relate to a second motion of the first user in the VR environment;
determine, based on the third data, that the second motion of the first user relates to a second symptom of the one or more mental illnesses;
determine, based on the third data, a severity level of the second symptom; and
control the wearable device of the second user to generate a second haptic output corresponding to the severity level of the second symptom.

9. The device according to claim 8, wherein the third data represents that the first user picks up or gravitates towards the one or more objects in the VR environment.

10. The device according to claim 9, wherein in determining the severity level of the second symptom, the one or more processors are configured to:
determine, based on the number of the one or more objects picked up or gravitated towards by the first user, the severity level of the second symptom.

11. A method for diagnosing one or more mental illnesses using a virtual reality (VR) environment in which a first user and a second user are present, the method comprising:
receiving, by one or more processors, from a head worn device (HWD) of the first user, first data representing a first motion of the first user in the VR environment;
determining, by the one or more processors based on the first data, that the first motion of the first user relates to a first symptom of the one or more mental illnesses;
receiving, by the one or more processors, from the HWD of the first user, second data representing at least one of an intensity, a boundary, or a frequency of the first motion of the first user;
determining, by the one or more processors based on the second data, a severity level of the first symptom; and
controlling, by the one or more processors, a wearable device of the second user to generate a first haptic output corresponding to the severity level of the first symptom.

12. The method according to claim 11, wherein the VR environment includes a plurality of virtual spaces in each of which the first user selects and/or manipulates one or more objects during a play therapy session.

13. The method according to claim 11, further comprising:
casting from the HWD of the first user to a display device of the second user.

14. The method according to claim 11, wherein
the intensity of the first motion of the first user represents an intensity of the first user releasing, throwing, or hitting one or more objects in the VR environment.

15. The method according to claim 14, wherein determining the severity level of the first symptom comprises:
determining the severity level of the first symptom based at least on an gaze direction or a movement pattern of an eye of the first user while the user releases, throws, or hit the one or more objects in the VR environment.

16. The method according to claim 11, wherein
the boundary of the first motion of the first user represents a boundary of the first motion of the first user with respect to a location of at least one of a floor, a ceiling, or a wall of the VR environment.

17. The method according to claim 11, wherein
the frequency of the first motion of the first user represents a number of repeating the first motion of the first user within a predetermined time.

18. The method according to claim 11, further comprising:
receiving, from the HWD of the first user, third data representing one or more objects that relate to a second motion of the first user in the VR environment;
determining, based on the third data, that the second motion of the first user relates to a second symptom of the one or more mental illnesses;
determining, based on the third data, a severity level of the second symptom; and
controlling the wearable device of the second user to generate a second haptic output corresponding to the severity level of the second symptom.

19. The method according to claim 18, wherein the third data represents that the first user picks up or gravitates towards the one or more objects in the VR environment.

20. The method according to claim 19, wherein determining the severity level of the second symptom comprises:
determining, based on the number of the one or more objects picked up or gravitated towards by the first user, the severity level of the second symptom.
